(19) United States
(12) Patent Application Publication
(10) Pub. No.: US 2026/0207320 A1
(43) Pub. Date: Jul. 23, 2026

(54) MEDICAL IMPLANT DELIVERY DEVICES

(71) Applicants: Smith & Nephew, Inc., Memphis, TN (US); Smith & Nephew Asia Pacific Pte. Limited

(72) Inventor: Timothy J. Young, Upton, MA (US)

(73) Assignees: Smith & Nephew, Inc., Memphis, TN (US); Smith & Nephew Asia Pacific Pte. Limited, (SG)

(21) Appl. No.: 19/453,422

(22) Filed: Jan. 20, 2026

Related U.S. Application Data
(60) Provisional application No. 63/747,670, filed on Jan. 21, 2025.

(51) Int. Cl. A61F 2/08; A61L 31/04

(52) U.S. Cl. CPC A61F 2/0805; A61L 31/044; A61F 2002/0852

(57) ABSTRACT

A medical device for delivering an implant includes a delivery shaft, an implant secured relative to the delivery shaft, and one or more sutures fastened to the implant. A delivery sheath slides over the implant and accommodates the sutures during translation. The delivery sheath may include elongate slots or apertures to accommodate the sutures, allowing them to translate as the sheath withdraws. The sheath may also feature apertures permitting hydrating fluid to penetrate and hydrate the implant. Flexible arms extending distally from the delivery shaft may secure the implant. The sutures extend proximally from the implant and may pass from within the delivery sheath to outside the sheath. The implant may be a collagen scaffold or a reinforced collagen scaffold, with the sutures secured to a reinforcement structure within the collagen scaffold.

### CROSS REFERENCE TO RELATED APPLICATIONS

This claims the benefit of and priority to US Provisional Patent Application Serial No. 63/747,670, filed on January 21, 2025, the disclosure of which is incorporated herein by reference.

### TECHNICAL FIELD

The present disclosure pertains generally, but not by way of limitation, to orthopedic implants and methods of treatment. More particularly, the present disclosure relates to a tendon repair implant, such as one that is engineered for arthroscopic placement over or in the area of a full or partial thickness tear of the supraspinatus tendon of the shoulder

### BACKGROUND

With its complexity, range of motion and extensive use, a common soft tissue injury is damage to the rotator cuff or rotator cuff tendons. Damage to the rotator cuff is a potentially serious medical condition that may occur during hyperextension, from an acute traumatic tear or from overuse of the joint. Adequate procedures do not exist for repairing a partial thickness tear of less than 50% in the supraspinatus tendon. Current procedures attempt to alleviate impingement or make room for movement of the tendon to prevent further damage and relieve discomfort but do not repair or strengthen the tendon. Use of the still damaged tendon can lead to further damage or injury. There is an ongoing need to deliver and adequately position medical implants during an arthroscopic procedure in order to treat injuries to the rotator cuff, rotator cuff tendons, or other soft tissue or tendon injuries throughout a body.

### BRIEF SUMMARY

This disclosure provides design, material, manufacturing method, and use alternatives for implants used for soft tissue repairs such as but not limited to rotator cuff repairs. An example may be found in a medical device that is configured to deliver an implant. The medical device includes a delivery shaft and an implant that is secured relative to the delivery shaft. One or more sutures are coupled to the implant. A delivery sheath is slidingly disposed over the implant and is configured to accommodate the one or more sutures when the delivery sheath translates relative to the implant.

Alternatively or additionally, the one or more sutures may include a single suture that loops through implant with two loose ends trailing proximally from the implant.

Alternatively or additionally, the one or more sutures may include a first suture fastened to the implant and a second suture fastened to the implant at a location spaced from where the first suture is fastened to the implant. Each suture includes a loose end that trails proximally from the implant.

Alternatively or additionally, the one or more sutures may extend from a position within the delivery sheath to a position outside of the delivery sheath.

Alternatively or additionally, the one or more sutures may extend outside of the delivery shaft.

Alternatively or additionally, the one or more sutures may extend within the delivery shaft.

Alternatively or additionally, the delivery sheath may include one or more elongate slots for accommodating the one or more sutures.

Alternatively or additionally, the one or more elongate slots may allow the one or more sutures to translate relative to the delivery sheath as the delivery sheath is withdrawn proximally.

Alternatively or additionally, the delivery sheath may include a plurality of apertures that are configured to allow a hydrating fluid to penetrate the delivery sheath and hydrate the implant.

Alternatively or additionally, the medical device may further include one or more pairs of flexible arms that extend distally from the delivery shaft and secure the implant.

Alternatively or additionally, the implant may include a collagen scaffold.

Alternatively or additionally, the implant may include a reinforced collagen scaffold and the one or more sutures may be secured relative to a reinforcement within the reinforced collagen scaffold.

Another example may be found in a medical device that is configured to deliver an implant. The medical device includes a delivery sheath and an implant that is disposed within the delivery sheath. One or more sutures are coupled to the implant and trail proximally from the implant. The delivery sheath is configured to accommodate the one or more sutures trailing proximally from the implant.

Alternatively or additionally, the implant may be releasably secured to a delivery shaft extending within the delivery sheath.

Alternatively or additionally, the delivery sheath may include one or more openings to accommodate the one or more sutures trailing proximally from the implant.

Alternatively or additionally, the delivery sheath may be configured to allow hydration of the implant while the implant remains within the delivery sheath.

Alternatively or additionally, the implant includes a medial region and a lateral region, and the one or more sutures may be secured to the lateral region.

Another example may be found in a medical device that is configured to deliver an implant. The medical device includes a delivery shaft including a plurality of flexible arms and an implant that is releasably secured to the plurality of flexible arms. One or more sutures are coupled to the implant and trail therefrom. A delivery sheath is disposed over the implant and is configured to accommodate the one or more sutures.

Alternatively or additionally, the delivery sheath may be configured to allow the one or more sutures to pass from an interior of the delivery sheath to an exterior of the delivery sheath.

Alternatively or additionally, the one or more sutures may pass through an interior of the delivery shaft.

Alternatively or additionally, the one or more sutures may be looped shuttling sutures.

Alternatively or additionally, the implant may include a collagen scaffold.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 illustrates a cross-section of an anterior view of a shoulder of a patient;

FIG. 2 shows an example implant;

FIG. 3 shows an example implant;

FIG. 4 shows a portion of the example implant of FIG. 3;

FIG. 5 shows an example medical device;

FIG. 6 shows the example medical device of FIG. 5 with a delivery sheath partially withdrawn to partially expose the implant;

FIG. 7 shows the example medical device of FIG. 5 with the delivery sheath more fully withdrawn to expose the implant;

FIG. 8 shows the example medical device of FIG. 5 with the delivery sheath fully withdrawn to further expose the implant;

FIG. 9 shows an example medical device;

FIG. 10 shows an example medical device;

FIG. 11 shows an example bio-compatible implant;

FIG. 12 shows an example bio-compatible implant;

FIG. 13 shows an example bio-compatible implant; and

FIG. 14 shows an example bio-compatible implant.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

With its complexity, range of motion and extensive use, a common soft tissue injury is damage to the rotator cuff or rotator cuff tendons. Damage to the rotator cuff is a potentially serious medical condition that may occur during hyperextension, from an acute traumatic tear or from overuse of the joint. Current repair procedures may attempt to alleviate impingement or make room for movement of the tendon to prevent further damage and relieve discomfort but do not repair or strengthen the tendon. An accepted treatment for rotator cuff tears may include reattaching the torn tendon to the humeral head using sutures. Additionally, in treating rotator cuff tears, an accepted practice may also include the placement of a scaffold over the repaired tendon to mechanically reinforce the repaired tendon. Therefore, there is an ongoing need to deliver and adequately position medical implants during an arthroscopic procedure in order to treat injuries to the rotator cuff, rotator cuff tendons, or other soft tissue or tendon injuries throughout a body.

In some instances, a medical device is configured to deliver an implant. The medical device includes a delivery shaft and an implant that is secured relative to the delivery shaft. One or more sutures are coupled to the implant. A delivery sheath is slidingly disposed over the implant and is configured to accommodate the one or more sutures when the delivery sheath translates relative to the implant.

In some cases, the one or more sutures may include a single suture that loops through implant with two loose ends trailing proximally from the implant. In some cases, the one or more sutures may include a first suture fastened to the implant and a second suture fastened to the implant at a location spaced from where the first suture is fastened to the implant. Each suture includes a loose end that trails proximally from the implant. In some cases, the one or more sutures may extend from a position within the delivery sheath to a position outside of the delivery sheath. In some cases, the one or more sutures may extend outside of the delivery shaft. In some cases, the one or more sutures may extend within the delivery shaft. In some cases, the delivery sheath may include one or more elongate slots for accommodating the one or more sutures. In some cases, the one or more elongate slots may allow the one or more sutures to translate relative to the delivery sheath as the delivery sheath is withdrawn proximally. In some cases, the delivery sheath may include a plurality of apertures that are configured to allow a hydrating fluid to penetrate the delivery sheath and hydrate the implant. In some cases, the medical device may further include one or more pairs of flexible arms that extend distally from the delivery shaft and secure the implant. In some cases, the implant may include a collagen scaffold.

In some cases, the implant may include a reinforced collagen scaffold and the one or more sutures may be secured relative to a reinforcement within the reinforced collagen scaffold.

In some instances, a medical device is configured to deliver an implant. The medical device includes a delivery sheath and an implant that is disposed within the delivery sheath. One or more sutures are coupled to the implant and trail proximally from the implant. The delivery sheath is configured to accommodate the one or more sutures trailing proximally from the implant.

In some cases, the implant may be releasably secured to a delivery shaft extending within the delivery sheath. In some cases, the delivery sheath may include one or more openings to accommodate the one or more sutures trailing proximally from the implant. In some cases, the delivery sheath may be configured to allow hydration of the implant while the implant remains within the delivery sheath. In some cases, the implant includes a medial region and a lateral region, and the one or more sutures may be secured to the lateral region.

In some instances, a medical device is configured to deliver an implant. The medical device includes a delivery shaft including a plurality of flexible arms and an implant that is releasably secured to the plurality of flexible arms. One or more sutures are coupled to the implant and trail therefrom. A delivery sheath is disposed over the implant and is configured to accommodate the one or more sutures.

In some cases, the delivery sheath may be configured to allow the one or more sutures to pass from an interior of the delivery sheath to an exterior of the delivery sheath. In some cases, the implant may include a collagen scaffold.

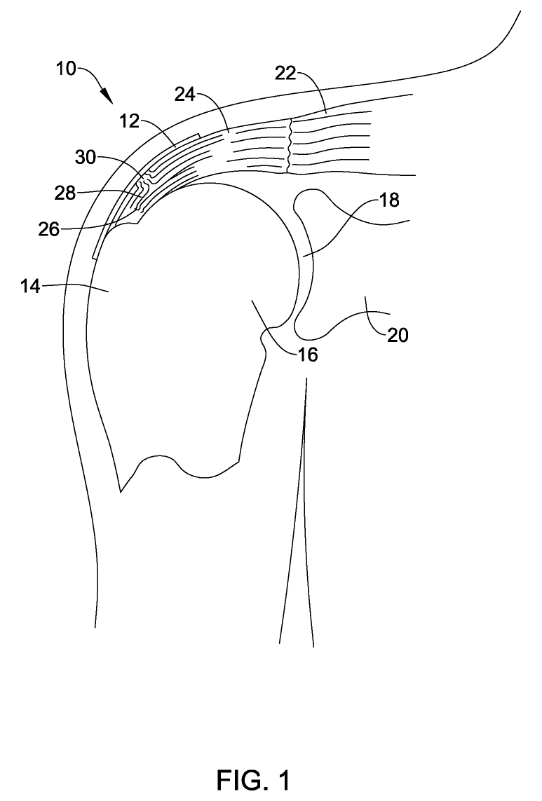

FIG. 1 shows a cross-sectional view of a shoulder 10 including an example bio-compatible implant 12. The shoulder 10 further shows a head 14 of a humerus 16 mating with a glenoid fossa 18 of scapula 20. The glenoid fossa 18 comprises a shallow depression in scapula 20. A supraspinatus tendon 22 is also shown. These muscles (along with others) control the movement of the humerus 16 relative to the scapula 20. A distal tendon 24 of the supraspinatus tendon 22 meets the humerus 16 at an insertion point 26.

In FIG. 1, the distal tendon 24 includes a damaged portion 28 located near the insertion point 26. The damaged portion 28 includes a tear 30 extending partially through the distal tendon 24. The tear 30 may be referred to as a partial thickness tear. In some cases, the tear 30 may be a full thickness tear. In some cases, the tear 30 may be a weakened tendon without a tear. The depicted partial thickness tear 30 is on the bursal side of the tendon, however, the tear may also be on the opposite or articular side of the distal tendon 24 and/or may include internal tears to the distal tendon 24 not visible on either surface. In other instances the tear 30 may be a full thickness tear.

FIG. 1 further illustrates that the bio-compatible implant 12 has been placed over the partial thickness tear 30. In this example, the bio-compatible implant 12 is placed on the bursal side of the tendon regardless of whether the tear is on the bursal side, articular side or within the tendon. Further, the bio-compatible implant 12 may overlay multiple tears.

Although one possible treatment site is described in the figures as being located in a shoulder joint, the bio-compatible implant 12 may be used at a variety of different treatment sites, such as the hip, knee, ankle, et. Furthermore, the bio-compatible implant 12 may be used for any of a variety of soft tissue repairs, such as but not limited to the Gluteus Medius, which is a large fan-shaped muscle located in the posterior hip, the Hip Capsule, which is also in the hip. The bio-compatible implant 12 may be used in treating soft tissue injuries in the knee, such as but not limited to ligaments such as the ACL (anterior cruciate ligament), MCL (medial collateral ligament) and the PCL (posterior cruciate ligament) and tendons such as the hamstring tendons, the quadriceps tendon and the patellar tendon. The bio-compatible implant 12 may be used in treating soft tissue injuries in the Achilles tendon. The bio-compatible implant 12 may be used in treating soft tissue injuries for any areas of the body that will accommodate the bio-compatible implant 12.

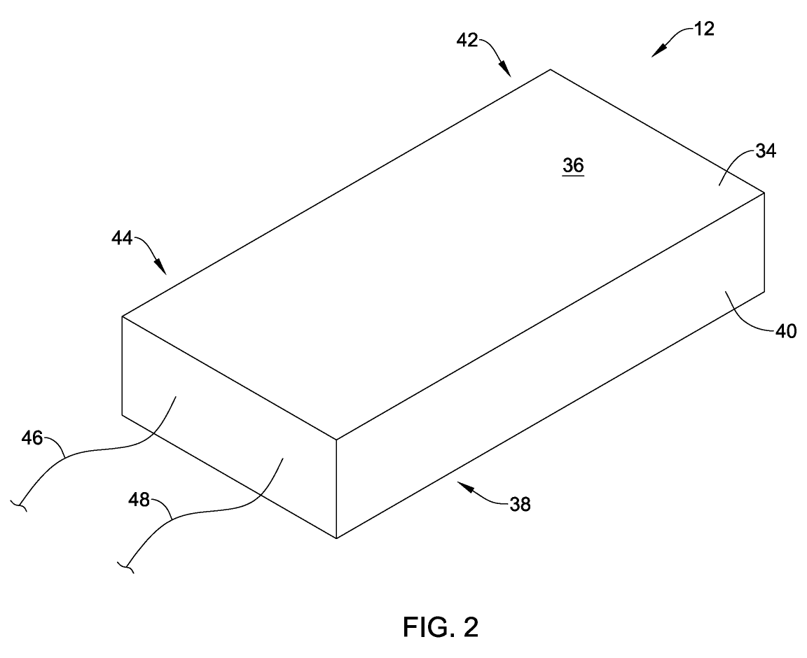

FIG. 2 is a schematic diagram of the example bio-compatible implant 12. The bio-compatible implant 12 may be used for any of a variety of soft tissue repairs, such as but not limited to the Gluteus Medius, which is a large fan-shaped muscle located in the posterior hip, the Hip Capsule, which is also in the hip. The bio-compatible implant 12 may be used in treating soft tissue injuries in the knee, such as but not limited to ligaments such as the ACL (anterior cruciate ligament), MCL (medial collateral ligament) and the PCL (posterior cruciate ligament) and tendons such as the hamstring tendons, the quadriceps tendon and the patellar tendon. The bio-compatible implant 12 may be used in treating soft tissue injuries in the Achilles tendon. The bio-compatible implant 12 may be used in treating soft tissue injuries for any areas of the body that will accommodate the bio-compatible implant 12.

For purposes of illustration, the bio-compatible implant 12 will be described herein with respect to rotator cuff repairs. The bio-compatible implant 12 may be considered as including a scaffold 34. In some instances, the scaffold 34 may define a first primary surface 36 and a second primary surface 38, and a periphery 40 that extends around between the first primary surface 36 and the second primary surface 38.

While drawn as rectilinear, it will be appreciated that this is merely illustrative, as the bio-compatible implant 12 may take any desired shape to best fit at a desired treatment site such as but not limited to a rotator cuff repair. In some cases, the bio-compatible implant 12 may be largely rectilinear, with rounded over corners, for example. In some cases, the bio-compatible implant 12 may be at least partially ovoid. The first primary surface 36 and the second primary surface 38 may be planar or curved. In some cases, the bio-compatible implant 12 may curve around a treatment site. These are just examples.

In some cases, the scaffold 34 may be a collagen scaffold, although other materials are contemplated. In some cases, the scaffold 34 is formed of collagen that has been dehydrated. The scaffold 34 may be formed as a fibrous collection of collagen fibers that define a porous scaffold with a large number of void spaces.

The collagen used to form the scaffold 34 may come from any of a variety of different sources. Collagen is a main structural protein in the extracellular matrix found in various connective tissues and thus can be obtained from various animals. For example, the collagen used to form the scaffold 34 may be bovine-based, i.e., from cows. In some cases, the collagen used to form the scaffold 34 may come from bovine tendon material. As an illustrative but non-limiting example, bovine Achilles tendon is digested down to highly purified collagen, and is reconstituted into a sheet by spinning the fibers around a mandrel. The scaffold 34 may be formed of collagen that has been obtained in other methods as well.

While the scaffold 34 may be formed having a variety of different porosity levels (defined as relative amount of void space to solid material), in some cases the scaffold 34 may have a porosity of at least 50 percent or more, at least 60 percent or more, at least 70 percent or more, or at least 80 percent or more. In some cases, the scaffold 34 may have a porosity of 60 percent to 90 percent, 70 percent to 90 percent, 80 percent to 90 percent, or 85 percent to 90 percent.

Another way to define the scaffold 34 is in terms of average pore size. Pore size refers to a diameter of voids, or empty spaces, formed within the scaffold 34. Average pore size, accordingly, refers to an average diameter of these voids. In some cases, the scaffold 34 may have an average pore size of 20 microns or greater, or 30 microns or greater, or 40 microns or greater, or 50 microns or greater, or 60 microns or greater, or 70 microns or greater, or 80 microns or greater, or 90 microns or greater. In some cases, the scaffold 34 may have an average pore size that is in a range of 100 microns to 500 microns, 100 microns to 400 microns, 100 microns to 300 microns,100 microns to 150 microns, or 200 microns to 400 microns, for example.

While not expressly shown in FIG. 2, the bio-compatible implant 12 may include a strengthening or reinforcing element that is adapted to provide strength to the soft tissue repair until tissue ingrowth into the bio-compatible implant 12 has occurred. It will be appreciated that the ultimate goal of the bio-compatible implant 12 is to facilitate tissue growth that provides long term strength and functionality to the soft tissue repair. The strengthening or reinforcing element, which may be biosorbable, is merely intended to provide additional strength to the bio-compatible implant 12 immediately after implantation of the bio-compatible implant 12. As the soft-tissue repair gains strength as a result of tissue ingrowth, the strengthening or reinforcing element becomes less important and in some cases may dissolve away.

The bio-compatible implant 12 may be dimensioned in accordance with its desired use. For example, if the bio-compatible implant 12 is intended for use in a rotator cuff repair, the bio-compatible implant 12 may have an overall length of 20 to 45 millimeters (mm) and an overall width of 20 to 40 mm. If the bio-compatible implant 12 is intended for use in an Achilles tendon repair, the bio-compatible implant 12 may have an overall length that is greater than 25 to 45 mm and an overall width that is less than 20 to 30 mm. In some cases, the bio-compatible implant 12 may have an overall thickness that is 1 to 4 mm. In some cases, the bio-compatible implant 12 may have an overall thickness that is about 2 mm. In defining these dimensions, it will be appreciated that the collagen scaffold 34 may not have a uniform thickness throughout the collagen scaffold 34, and the perimeter 40 may not be as evenly defined as shown in the rectilinear representation of FIG. 2. These dimensions may be considered as being averages, for example.

When the bio-compatible implant 12 is implanted as part of a procedure treating a rotator cuff tear, it will be appreciated that the bio-compatible implant 12 is implanted in such a way that the bio-compatible implant 12 will include a medial portion 42 that may be secured to a tendon and an opposing lateral portion 44 that may be secured to bone using a bone anchor such as a self-tapping or a non-self tapping HEALICOIL KNOTLESS™ commercially available from Smith & Nephew, for example. Other bone anchors may also be used. In some cases, one or more sutures may be coupled to the bio-compatible implant 12. In some cases, the one or more sutures may be slidingly disposed within the bio-compatible implant 12. In some cases, the one or more sutures may be tied in place, and thus may be considered as being fixed or secured in place relative to the bio-compatible implant 12. As shown, a first suture 46 and a second suture 48 are shown extending from the lateral portion 44 of the bio-compatible implant 12. In some cases, the bio-compatible implant 12 may include only a single suture extending therefrom. In some cases, the bio-compatible implant 12 may include three or more sutures extending therefrom. One or more of the first suture 46 and the second suture 48 may be used to secure the bio-compatible implant 12 to bone using one or more bone anchors.

In some cases, the first suture 46 and the second suture 48 (as well as any additional sutures not shown) may be coupled to the bio-compatible implant 12 when the bio-compatible implant 12 is formed. In some cases, the first suture 46 and the second suture 48 (as well as any additional sutures not shown) may be coupled to the bio-compatible implant 12 by using a needle or similar structure to weave the first suture 46 and/or the second suture 48 back and forth within the bio-compatible implant 12. The first suture 46 and the second suture 48 may be formed of any biocompatible material commonly used for sutures. The first suture 46 and/or the second suture 48 may be made from either absorbable or non-absorbable materials. Absorbable sutures break down naturally in the body through enzymatic degradation or hydrolysis and include materials like polyglycolic acid, polylactic acid, poliglecaprone, and polydioxanone (synthetic options), as well as catgut derived from purified collagen (natural option). Non-absorbable sutures remain in tissue indefinitely unless removed and include synthetic materials such as polypropylene, nylon, polyester, and polyethylene, along with natural materials like silk and stainless steel.

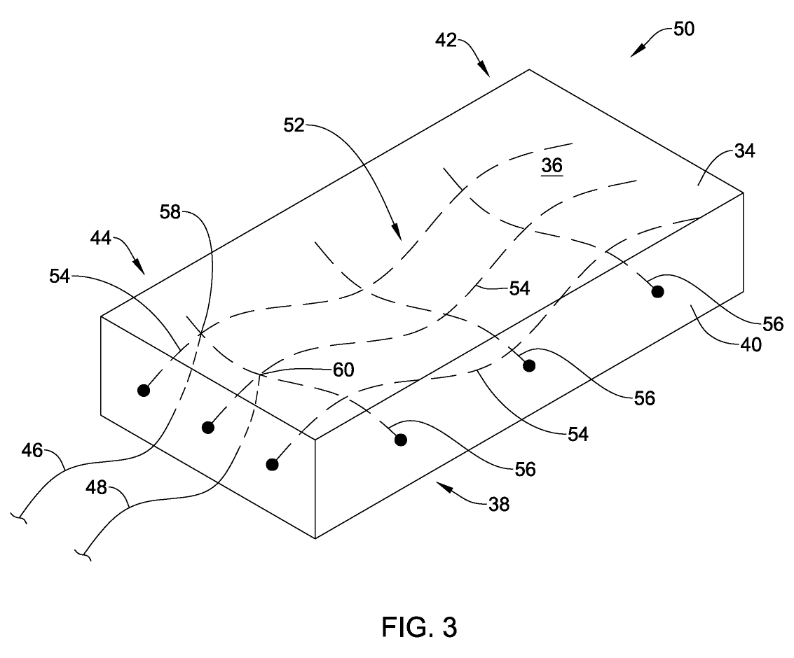

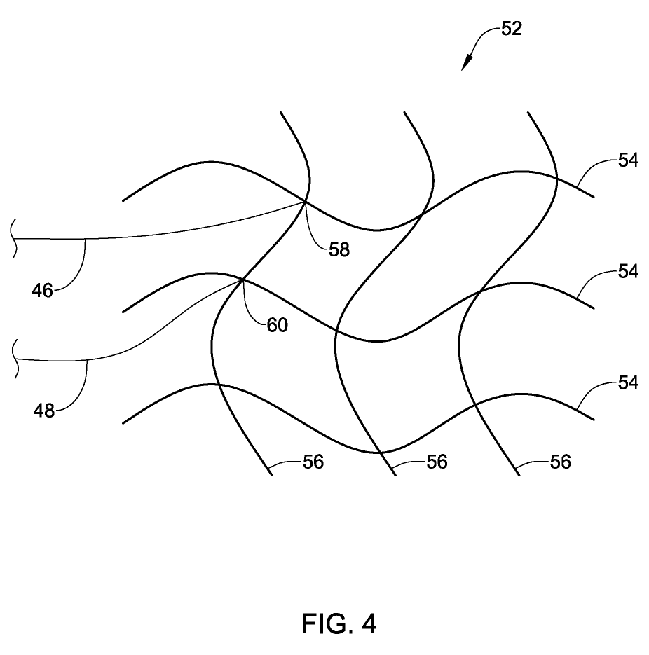

In some cases, the bio-compatible implant 12 may include a reinforcing material that provides additional strength to the bio-compatible implant 12. FIG. 3 is a schematic diagram of an example bio-compatible implant 50 that is essentially the same as the bio-compatible implant 12, but includes a reinforcing structure 52 that is disposed within the bio-compatible implant 50. In FIG. 3, the reinforcing structure 52 is shown in phantom while FIG. 4 shows the reinforcing structure 52 without the collagen scaffold 34. The reinforcing structure 52 may take any of a variety of different forms. As shown, the reinforcing structure 52 is a woven structure, including several (three are shown) filaments 54 that extend generally in a lengthwise direction within the collagen scaffold 34 and several (three are shown) filaments 56 that extend generally orthogonally to the filaments 54. In some cases, the first suture 46 may extend into the bio-compatible implant 50 and may engage the reinforcing structure 52 at a connection point 58. In some cases, the connection point 58 may correspond to a connection between one of the filaments 56 and one of the filaments 58. In some cases, the second suture 48 may extend into the bio-compatible implant 50 and may engage the reinforcing structure 52 at a connection point 60. In some cases, the connection point 60 may correspond to a connection between one of the filaments 56 and one of the filaments 58. In some cases, additional strength is provided by engaging the first suture 46 and the second suture 48 with the reinforcing structure 52. The reinforcing structure 52 may be formed of any of a variety of different materials. In some cases, the reinforcing structure 52 may be formed of the same material that is used to form the first suture 46 and the second suture 48, for example.

The bio-compatible implant 50 may be used for any of a variety of soft tissue repairs, such as but not limited to the Gluteus Medius, which is a large fan-shaped muscle located in the posterior hip, the Hip Capsule, which is also in the hip. The bio-compatible implant 50 may be used in treating soft tissue injuries in the knee, such as but not limited to ligaments such as the ACL (anterior cruciate ligament), MCL (medial collateral ligament) and the PCL (posterior cruciate ligament) and tendons such as the hamstring tendons, the quadriceps tendon and the patellar tendon. The bio-compatible implant 50 may be used in treating soft tissue injuries in the Achilles tendon. The bio-compatible implant 50 may be used in treating soft tissue injuries for any areas of the body that will accommodate the bio-compatible implant 50.

For purposes of illustration, the bio-compatible implant 50 will be described herein with respect to rotator cuff repairs. The bio-compatible implant 50 may be considered as including the scaffold 34. In some instances, the scaffold 34 may define the first primary surface 36 and the second primary surface 38, with the periphery 40 extending around between the first primary surface 36 and the second primary surface 38.

While drawn as rectilinear, it will be appreciated that this is merely illustrative, as the bio-compatible implant 50 may take any desired shape to best fit at a desired treatment site such as but not limited to a rotator cuff repair. In some cases, the bio-compatible implant 50 may be largely rectilinear, with rounded over corners, for example. In some cases, the bio-compatible implant 50 may be at least partially ovoid. The first primary surface 36 and the second primary surface 38 may be planar or curved. In some cases, the bio-compatible implant 50 may curve around a treatment site. These are just examples.

In some instances, delivery of a bio-compatible implant 12, 50 (e.g., a sheet-like implant) to a target site of a patient may require a physician to create an incision in the patient sufficient to access the target implant site. After creating this “access site,” the physician may insert an implant delivery system through the access site and position the distal end of the implant delivery system adjacent the target implant site. The physician may then manipulate the implant delivery system to deploy an implant out of a delivery sheath adjacent the target implant site.

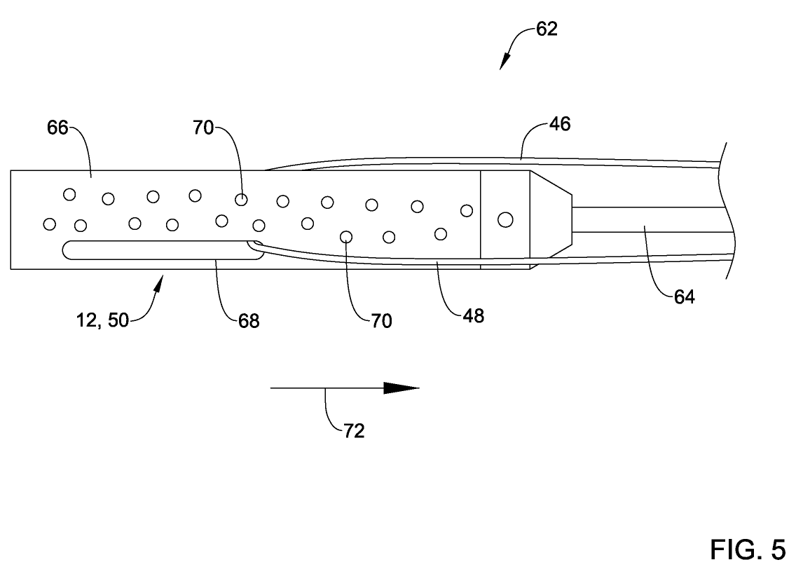

FIG. 5 is a side view of an example medical device 62 that may be used for delivering the bio-compatible implant 12 or the bio-compatible implant 50. The implant itself is not visible in FIG. 5, but the first suture 46 and the second suture 48 extending from the implant are visible. The example medical device 62 includes a delivery shaft 64 and a delivery sheath 66. While not visible, the medical device 62 includes the bio-compatible implant 12 or the bio-compatible implant 50. In some cases, as will be discussed, the delivery shaft 64 may include structure that helps to secure the bio-compatible implant 12, 50 relative to the delivery shaft 64. As shown, the first suture 46 and the second suture 48 extend proximally (such as to an unseen handle) outside of the delivery shaft 64. In some cases, the first suture 46 and the second suture 48 may instead extend within a lumen (not shown) formed within the delivery shaft 64.

In some cases, the delivery sheath 66 may be configured to accommodate the first suture 46 and the second suture 48 extending proximally from the bio-compatible implant 12, 50. In some cases, the delivery sheath 66 includes one or more elongate slots 68 (only one is visible) that extend along the delivery sheath 66. In some cases, there is a second elongate slot 68 that extends along the delivery sheath 66 but is not visible in this view. As an example, the first elongate slot 68 and the second elongate slot 68 may be circumferentially 180 degrees apart. In some cases, the first elongate slot 68 and the second elongate slot 68 may be circumferentially separated by less than 180 degrees (or more than 180 degrees, depending on perspective).

In some cases, the delivery sheath 66 may be configured to accommodate hydrating the bio-compatible implant 12, 50 prior to implantation. In some cases, the delivery sheath 66 includes a plurality of apertures 70 (not all are labeled) that allows a hydrating solution such as saline to flow into an interior of the delivery sheath 66 and thus hydrate the bio-compatible implant 12, 50. In some cases, the bio-compatible implant 12, 50 may be at least partially hydrated by a hydrating solution entering the delivery sheath 66 via an open end thereof. In some cases, the bio-compatible implant 12, 50 may be hydrated while the bio-compatible implant 12, 50 is secured relative to the delivery shaft 64 and is disposed within the delivery sheath 66 by soaking the medical device 62 within a basin of hydrating solution. As shown in FIG. 5, the delivery sheath 66 may be considered as being in a fully distal position in which the bio-compatible implant 12, 50 is entirely disposed within the delivery sheath 66.

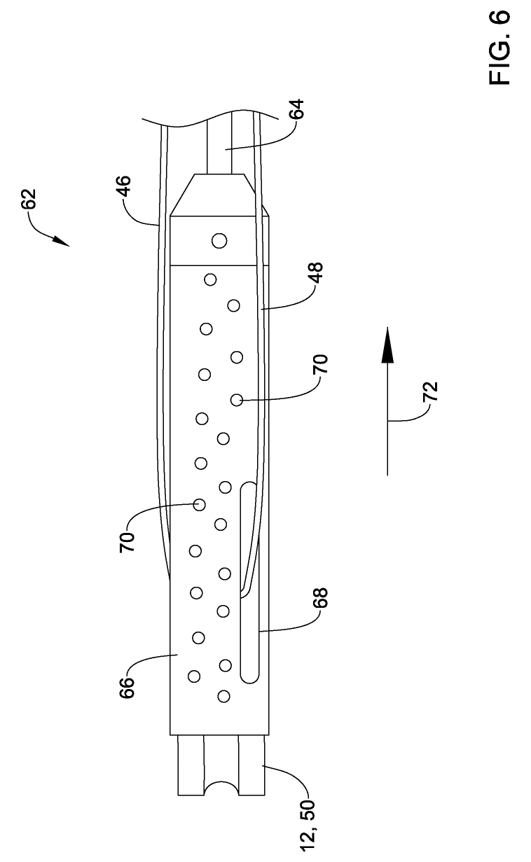

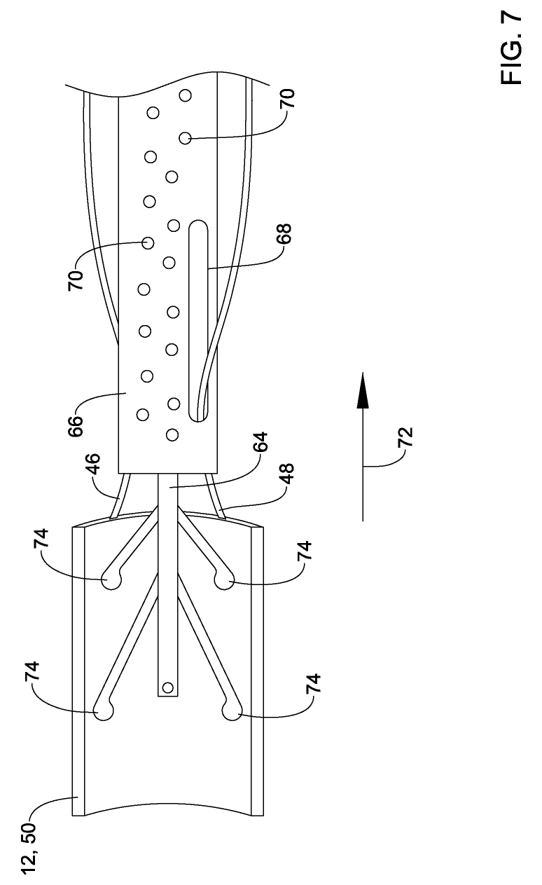

In order to expose and deliver the bio-compatible implant 12, 50, the delivery sheath 66 may be moved proximally relative to the rest of the medical device 62, in a direction indicated by an arrow 72. In some cases, the bio-compatible implant 12 may be pushed out of the delivery sheath 66. In FIG. 6, the delivery sheath 66 has been moved a short distance in the direction indicated by the arrow 72 while the rest of the medical device 62 is held steady. As a result, the bio-compatible implant 12, 50 is beginning to extend distally out of the delivery sheath 66. Continuing to move the delivery sheath 66 in the direction indicated by the arrow 72 results in a configuration shown in FIG. 7. As seen in FIG. 7, the bio-compatible implant 12, 50 is now fully exposed, and is no longer constrained in a rolled or folded configuration by the delivery sheath 66. The first suture 46 and the second suture 48 may now be seen as extending proximally (or laterally) from the bio-compatible implant 12, 50.

In some cases, the delivery shaft 64 may include one or more telescoping portions. In some cases, the medical device 62 includes a plurality of flexible arms 74 that extend from the delivery shaft 64 and that releasably secure the bio-compatible implant 12, 50 relative to the delivery shaft 64. In some cases, the plurality of flexible arms 74 may be formed of a shape memory material such as nitinol, which allows the plurality of flexible arms 74 to possibly deform from a remembered shape when the bio-compatible implant 12, 50 is rolled or folded within the delivery sheath 66. In some cases, the plurality of flexible arms 74 may be arranged in pairs of flexible arms 74, as shown. In some cases, the plurality of flexible arms 74 may be configured to attach to the bio-compatible implant 12, 50 using a friction fit, elastic compression members, pins, springs, and/or other suitable coupling techniques and components.

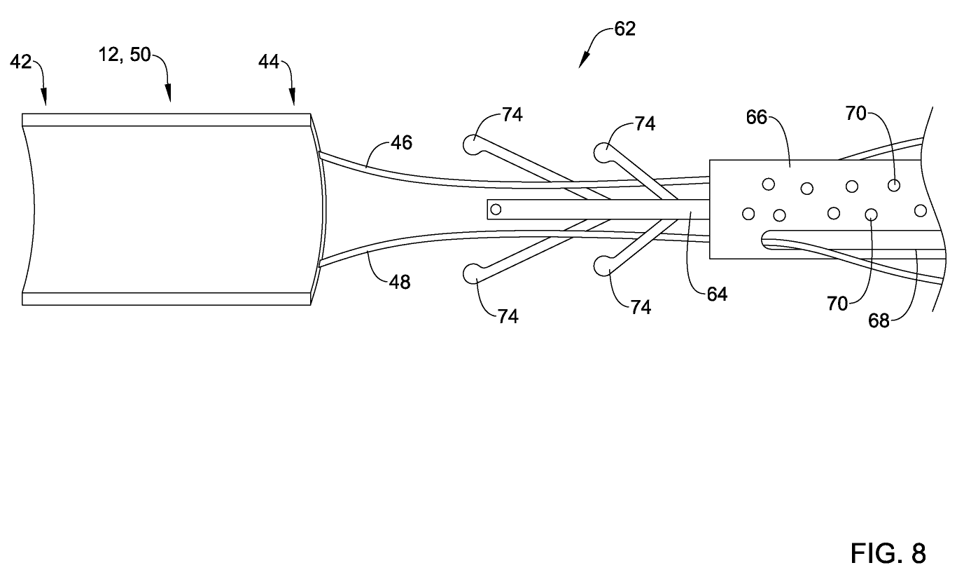

Continuing to move the delivery sheath 66 in the direction indicated by the arrow 72 will result in the bio-compatible implant 12, 50 being free of the plurality of flexible arms 74, as shown for example in FIG. 8. At this point, the medial portion 42 of the bio-compatible implant 12, 50 may be secured relative to a tendon. In some cases, the medial portion 42 of the bio-compatible implant 12, 50 may have already been secured relative to a tendon. In some cases, the first suture 44 and/or the second suture 46 may be used to secure the lateral portion 44 of the bio-compatible implant 12, 50 to bone (such as the shoulder 10). In some cases, the first suture 44 and/or the second suture 46 may be used to secure the bio-compatible implant 12, 50 to the bone via one or more bone anchors. In some cases, as an alternative, lateral sutures may be attached prior to medial fixation.

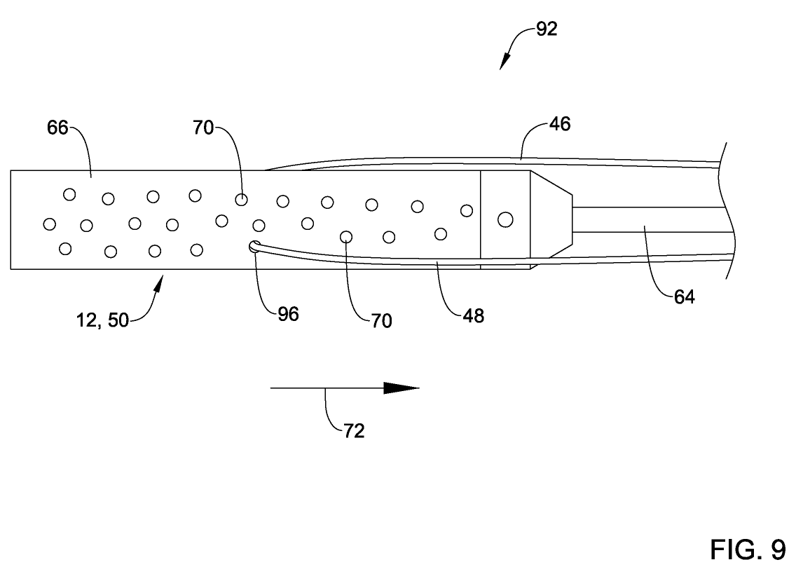

FIG. 9 is a side view of an example medical device 92 that may be used for delivering the bio-compatible implant 12 or the bio-compatible implant 50. The implant itself is not visible in FIG. 9, but the first suture 46 and the second suture 48 extending from the implant are visible. The example medical device 92 includes the delivery shaft 64 and the delivery sheath 66, much like the medical device 62 shown in FIG. 5. While not visible, the medical device 92 includes the bio-compatible implant 12 or the bio-compatible implant 50. In some cases, as will be discussed, the delivery shaft 64 may include structure that helps to secure the bio-compatible implant 12, 50 relative to the delivery shaft 64. As shown, the first suture 46 and the second suture 48 extend proximally (such as to an unseen handle) outside of the delivery shaft 64. In some cases, the first suture 46 and the second suture 48 may instead extend within a lumen (not shown) formed within the delivery shaft 64.

In some cases, the delivery sheath 66 may be configured to accommodate the first suture 46 and the second suture 48 extending proximally from the bio-compatible implant 12, 50. In some cases, the delivery sheath 66 includes one or openings 96 (only one is visible) that extend along the delivery sheath 66. In some cases, there is a second elongate slot 68 that extends along the delivery sheath 66 but is not visible in this view. As an example, the first elongate slot 68 and the second elongate slot 68 may be circumferentially 180 degrees apart. In some cases, the first elongate slot 68 and the second elongate slot 68 may be circumferentially separated by less than 180 degrees (or more than 180 degrees, depending on perspective). In some cases, the delivery sheath 66 may be configured to accommodate hydrating the bio-compatible implant 12, 50 prior to implantation. In some cases, the delivery sheath 66 includes a plurality of apertures 70 (not all are labeled) that allows a hydrating solution such as saline to flow into an interior of the delivery sheath 66 and thus hydrate the bio-compatible implant 12, 50. In some cases, the one or more openings 96 may be larger than the plurality of apertures 70. In some cases, the one or more openings 96 may simply be one or more of the plurality of apertures 70. In some cases, the bio-compatible implant 12, 50 may be at least partially hydrated by a hydrating solution entering the delivery sheath 66 via an open end thereof. In some cases, the bio-compatible implant 12, 50 may be hydrated while the bio-compatible implant 12, 50 is secured relative to the delivery shaft 64 and is disposed within the delivery sheath 66 by soaking the medical device 62 within a basin of hydrating solution.

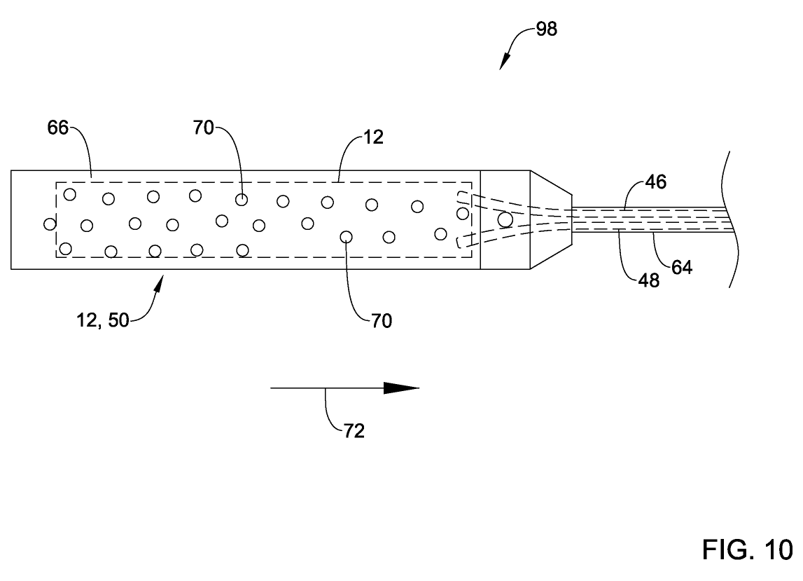

FIG. 10 is a side view of an example medical device 98 that may be used for delivering the bio-compatible implant 12 or the bio-compatible implant 50. In some cases, the bio-compatible implant 12 is rolled up within the delivery sheath 66. The first suture 46 and the second suture 48 extending from the bio-compatible implant 12 are visible. The example medical device 62 includes the delivery shaft 64. While not visible, the medical device 62 includes the bio-compatible implant 12 or the bio-compatible implant 50. In some cases, as will be discussed, the delivery shaft 64 may include structure that helps to secure the bio-compatible implant 12, 50 relative to the delivery shaft 64. As shown, the first suture 46 and the second suture 48 extend proximally (such as to an unseen handle) inside of the delivery shaft 64.

In some cases, the delivery sheath 66 may be configured to accommodate hydrating the bio-compatible implant 12, 50 prior to implantation. In some cases, the delivery sheath 66 includes a plurality of apertures 70 (not all are labeled) that allows a hydrating solution such as saline to flow into an interior of the delivery sheath 66 and thus hydrate the bio-compatible implant 12, 50. In some cases, the bio-compatible implant 12, 50 may be at least partially hydrated by a hydrating solution entering the delivery sheath 66 via an open end thereof. In some cases, the bio-compatible implant 12, 50 may be hydrated while the bio-compatible implant 12, 50 is secured relative to the delivery shaft 64 and is disposed within the delivery sheath 66 by soaking the medical device 62 within a basin of hydrating solution.

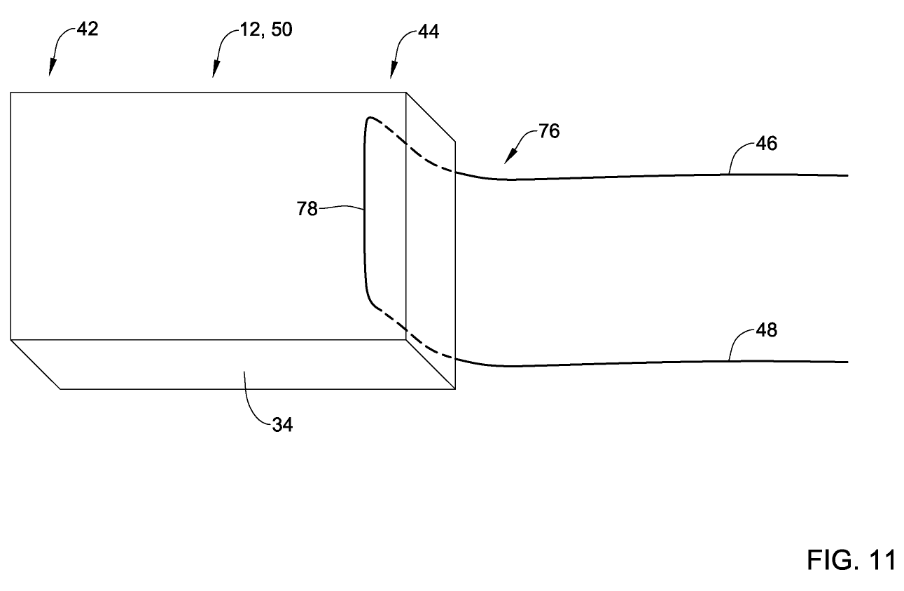

As shown in FIGS. 2 through 10, the first suture 46 and the second suture 48 are separate sutures that are each coupled with or secured to the bio-compatible implant 12, 50. FIG. 11 provides an example in which a single suture 76 loops through the bio-compatible implant 12, 50 to form the separate first suture 46 and the second suture 48. In some cases, having a portion 78 of the single suture 76 that loops through the bio-compatible implant 12, 50 helps to secure the single suture 76 relative to the bio-compatible implant 12, 50. In some cases, the portion 78 extends outside of a bottom (when implanted) of the scaffold 34. In some cases, the single suture 76 is free to slide back and forth relative to the In some cases, as shown, the single suture 76 may loop through the lateral portion 44 of the bio-compatible implant 12, 50. In some cases, the single suture 76 may instead extend further into the bio-compatible implant 12, 50 such that the portion 78 extend into the medial portion 42 as well. In some cases, the single suture 76 may instead be part of, or interact with, the reinforcing structure 52 shown in FIGS. 3 and 4.

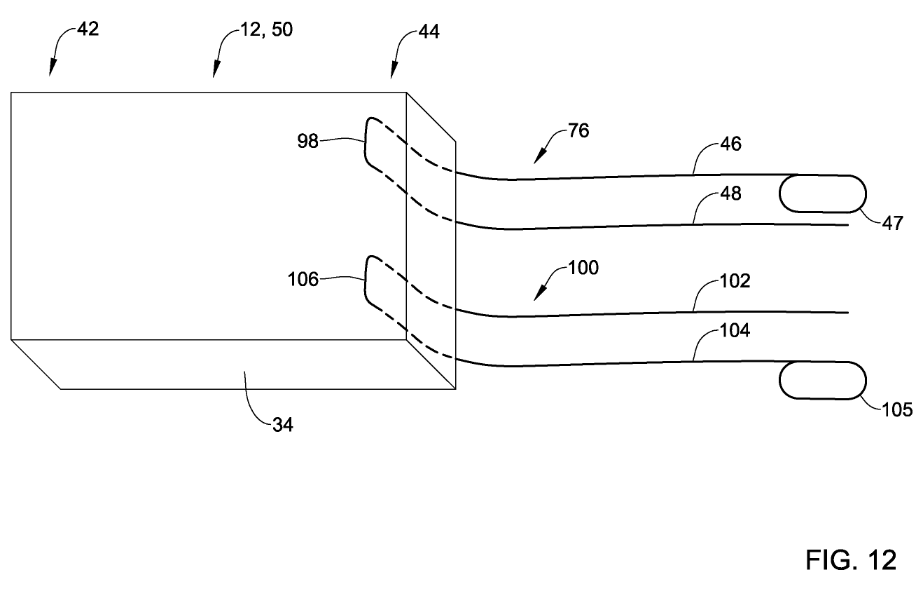

FIG. 12 provides an example that includes the single suture 76 as well as a second suture 100. The single suture 76 loops through the bio-compatible implant 12, 50 to form the separate first suture 46 and the second suture 48. In some cases, having a portion 78 of the single suture 76 that loops through the bio-compatible implant 12, 50 helps to secure the single suture 76 relative to the bio-compatible implant 12, 50. In some cases, the portion 78 extends outside of a top of the scaffold 34. In some cases, the single suture 76 is free to slide back and forth relative to the scaffold 34. In some cases, as shown, the single suture 76 may loop through the lateral portion 44 of the bio-compatible implant 12, 50. In some cases, the single suture 76 may instead extend further into the bio-compatible implant 12, 50 such that the portion 78 extend into the medial portion 42 as well. In some cases, the single suture 76 may instead be part of, or interact with, the reinforcing structure 52 shown in FIGS. 3 and 4.

In some cases, a second suture 100 loops through the bio-compatible implant 12, 50 to form a separate first suture 102 and a second suture 104. In some cases, having a portion 106 of the second suture 100 that loops through the bio-compatible implant 12, 50 helps to secure the second suture 100 relative to the bio-compatible implant 12, 50. In some cases, the portion 106 extends outside of a bottom (when implanted) of the scaffold 34. In some cases, the second suture 100 is free to slide back and forth relative to the scaffold 34. In some cases, as shown, the second suture 100 may loop through the lateral portion 44 of the bio-compatible implant 12, 50. In some cases, the second suture 100 may instead extend further into the bio-compatible implant 12, 50 such that the portion 78 extend into the medial portion 42 as well. In some cases, the second suture 100 may instead be part of, or interact with, the reinforcing structure 52 shown in FIGS. 3 and 4.

In some cases, the first suture 46 may include or otherwise define a loop 47 and the second suture 48 may include or otherwise define a loop 105. In some cases, the loop 47 and/or the loop 105 may be used for fixation to an anchor or to shuttle a repair suture from a pre-loaded suture implant such as Healicoil, QFIX, or GFIX Knotless), among others.

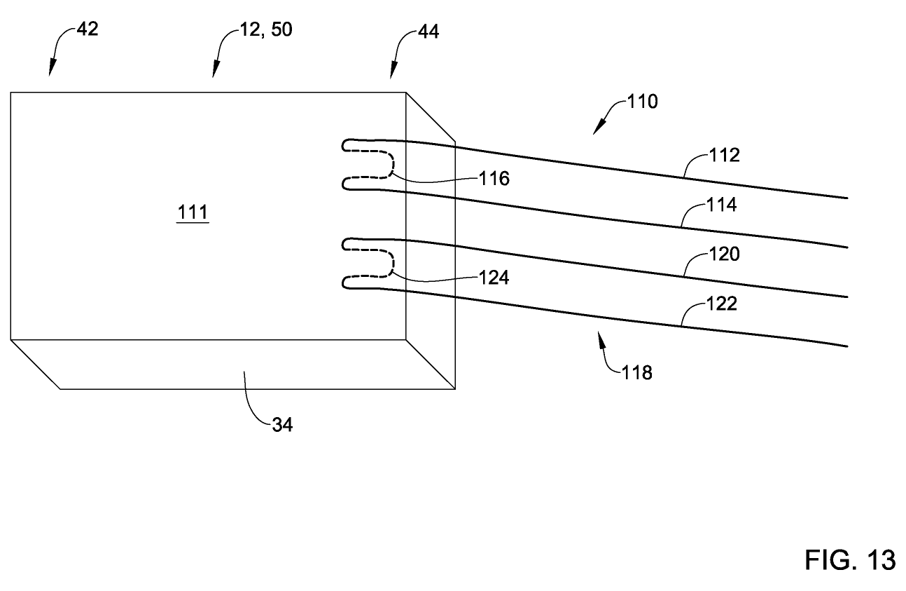

FIG. 13 provides an example that includes a first suture 110 and a second suture 118. The first suture 110 extends into a surface 111 of the scaffold 34 such that a loop portion 116 of the first suture 110 is disposed within an interior of the scaffold 34 and includes a first trailing suture 112 and a second trailing suture 114. The second suture 118 extends into the surface 111 of the scaffold 34 such that a loop portion 124 of the second suture 118 is disposed within an interior of the scaffold 34 and includes a first trailing suture 120 and a second trailing suture 122. In some cases, as shown, the first suture 110 and/or the second suture 118 may loop through the lateral portion 44 of the bio-compatible implant 12, 50. In some cases, the first suture 110 and/or the second suture 118 may instead extend further into the bio-compatible implant 12, 50 such that the first suture 110 and/or the second suture 118 loop through the medial portion 42.

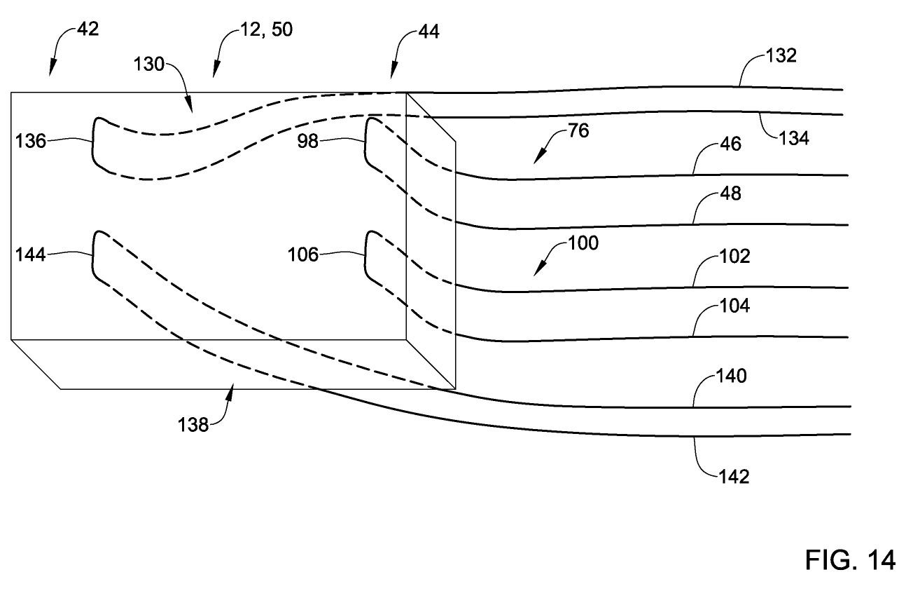

FIG. 14 provides an example that includes the single suture 76 and the second suture 100 looping through the lateral portion 44 of the scaffold 34 as well as a third suture 130 and a fourth suture 138 that each loop through the medial portion 42 of the scaffold 34. The single suture 76 loops through the bio-compatible implant 12, 50 to form the separate first suture 46 and the second suture 48 as well as the loop portion 78. The second suture 100 loops through the bio- compatible implant 12, 50 to form the separate first suture 102 and the second suture 104 as well as the loop portion 106. The third suture 130 loops through the scaffold 34 to form a separate suture 132 and a suture 134 as well as a loop portion 136 therebetween. The fourth suture 138 loops through the scaffold 34 to form a separate suture 140 and a suture 144 as well as a loop portion 144 therebetween.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure’s scope is, of course, defined in the language in which the appended claims are expressed.

What is claimed is:

1. A medical device configured to deliver an implant, the medical device comprising:
a delivery shaft;
an implant secured relative to the delivery shaft;
one or more sutures coupled to the implant; and
a delivery sheath slidingly disposed over the implant, the delivery sheath configured to accommodate the one or more sutures when the delivery sheath translates relative to the implant.

2. The medical device of claim 1, wherein the one or more sutures comprise a single suture that loops through implant with two loose ends trailing proximally from the implant.

3. The medical device of claim 1, wherein the one or more sutures comprises a first suture fastened to the implant and a second suture fastened to the implant at a location spaced from where the first suture is fastened to the implant, each suture with a loose end trailing proximally from the implant.

4. The medical device of claim 1, wherein the one or more sutures extend from a position within the delivery sheath to a position outside of the delivery sheath.

5. The medical device of claim 1, wherein the one or more sutures extend outside of the delivery shaft.

6. The medical device of claim 1, wherein the one or more sutures extend within the delivery shaft.

7. The medical device of claim 1, wherein the delivery sheath comprises one or more openings for accommodating the one or more sutures.

8. The medical device of claim 7, wherein the one or more openings allow the one or more sutures to translate relative to the delivery sheath as the delivery sheath is withdrawn proximally.

9. The medical device of claim 1, wherein the delivery sheath comprises a plurality of apertures configured to allow a hydrating fluid to penetrate the delivery sheath and hydrate the implant.

10. The medical device of claim 1, further comprising one or more pairs of flexible arms extending distally from the delivery shaft, the one or more pairs of flexible arms secured to the implant.

11. The medical device of claim 1, wherein the implant comprises a collagen scaffold.

12. The medical device of claim 1, wherein the implant comprises a reinforced collagen scaffold and the one or more sutures are secured relative to a reinforcement within the reinforced collagen scaffold.

13. A medical device configured to deliver an implant, the medical device comprising:
a delivery sheath;
an implant disposed within the delivery sheath; and
one or more sutures coupled to the implant and trailing proximally from the implant;
wherein the delivery sheath is configured to accommodate the one or more sutures trailing proximally from the implant.

14. The medical device of claim 13, wherein the implant is releasably secured to a delivery shaft extending within the delivery sheath.

15. The medical device of claim 13, wherein the delivery sheath comprises one or more openings to accommodate the one or more sutures trailing proximally from the implant.

16. The medical device of claim 13, wherein the delivery sheath is configured to allow hydration of the implant while the implant remains within the delivery sheath.

17. The medical device of claim 13, wherein the implant includes a medial region and a lateral region, and the one or more sutures are secured to the lateral region.

18. A medical device configured to deliver an implant, the medical device comprising:
a delivery shaft including a plurality of flexible arms;
an implant releasably secured to the plurality of flexible arms;
one or more sutures coupled to the implant and trailing therefrom; and
a delivery sheath disposed over the implant, the delivery sheath configured to accommodate the one or more sutures.

19. The medical device of claim 18, wherein the delivery sheath is configured to allow the one or more sutures to pass from an interior of the delivery sheath to an exterior of the delivery sheath.

20. The medical device of claim 18, wherein the implant comprises a collagen scaffold.
